(19) United States
(12) Patent Application Publication
(10) Pub. No.: US 2016/0051161 A1
(43) Pub. Date: Feb. 25, 2016

(54) METHOD FOR LOCATING A BRAIN ACTIVITY ASSOCIATED WITH A TASK

(71) Applicant: Commissariat a L'Energie Atomique et aux Energies Alternatives, Paris (FR)

(72) Inventors: Etienne LABYT, Saint Martin le Vinoux (FR); Pierre DURAND, La Tranche (FR)

(73) Assignee: Commissariat a L'Energie Atomique et aux Energies Alternatives, Paris (FR)

(21) Appl. No.: 14/810,974

(22) Filed: Jul. 28, 2015

(30) Foreign Application Priority Data
Aug. 22, 2014 (FR) 14 57947

(51) Int. Cl. A61B 5/04; A61B 5/00; A61B 5/0476

(52) U.S. Cl. CPC A61B 5/04012; A61B 5/04008; A61B 5/0476; A61B 5/7235

(57) ABSTRACT

The invention relates to a method for estimating the brain activity, from physiological signals, in particular magnetoencephalographic or electroencephalographic surfaces, which has, in certain predetermined areas of the cortex, considered as areas of interest, an improved accuracy with respect to other areas of the gridding. It enables a more accurate estimation to be obtained in the areas of the brain intended to be subjected to a particular treatment, for example to accommodate cortical electrodes.

### TECHNICAL FIELD

The present invention generally relates to the estimation of the electrical activity in a human or animal tissue, and more particularly the localisation of a brain activity from physiological signals, obtained by magnetoencephalography or by electroencephalography. The invention especially applies to the field of functional neural imaging and direct neural control.

### STATE OF PRIOR ART

Functional neural imaging methods are conventionally divided into those based on the metabolic activity such as functional magnetic resonance imaging (fMRI), representing the hemodynamic answer, or positron emission tomography (PET), representing modifications in the blood flow, and methods based on the electrophysiological activity, such as electroencephalography (EEG), measuring the electrical brain activity by means of electrodes placed on the subject's scalp, electrocorticography (ECoG) measuring the brain activity by means of electrodes directly placed on the cortical surface, or magnetoencephalography (MEG) measuring the magnetic fields related to the electrical activity in the brain.

The EEG and MEG functional imaging methods have a better time resolution than the fMRI and TEP methods. Furthermore, they are non-invasive unlike electrocorticography. Finally, magnetoencephalography is particularly interesting in that the magnetic signals created by the currents in the brain (mainly ionic currents in dendrites during the synaptic transmission), undergo little or no distortion when they propagate through the cranium.

Besides, electroencephalography and magnetoencephalography are presently the object of considerable research for their potential applications to the direct neural control. Direct neural control or BCI (Brain Computer Interface) enables a direct communication to be established between the brain of a user and an external device (computer, electronic system, effector) with no muscle mediation. Direct neural control uses the association of one or more mental tasks (action imagined by the subject) with one or more controls of the external device. Thus, a hand action imagined by the subject can be associated with a movement of a cursor on a computer screen or the motion of an effector. This technique is very promising especially for people suffering from paralysis.

Whether in the field of functional neural imaging or that of direct control, different methods have been developed to locate a brain activity associated with an (imagined or performed) task from physiological signals acquired by a plurality of sensors. Thus, in the case of EEG, the sensors are electrodes enabling electric potential differences to be acquired at the scalp surface. In the case of MEG, the sensors (SQUIDs) can be magnetometers able to measure the intensity of the magnetic fields and/or planar (or axial) gradiometers able to measure the magnetic field gradient in a given plane. For example, an MEG equipment can combine, in a same location, three simple sensors or even more: a precision magnetometer measuring the intensity and orientation of the magnetic field at a point and two planar gradiometers, perpendicular to each other, measuring two components of the magnetic field gradient at this point.

In any case, the object of the abovementioned methods is to locate the brain activity sources from signals acquired by the different sensors. More precisely, if a gridding of the cortex into M elementary areas is performed and if x is a vector (of a size M) representative of electric current densities (or source signals) in the different elementary areas and y is a vector (of a size N) representative of the signals acquired by the different sensors, we have the matrix relationship:

y=Ax+b (1)

where A is a matrix of a size N×M, referred to as a lead field matrix which is a function of the considered elementary area and b is a noise sample vector of a size N.

These M elementary areas are distributed at the cortex surface, according to a predetermined gridding. The neuronal signal is then estimated in M representative points of these elementary areas. These M points are determined as a function of the gridding. It can be the centre or the vertices of each square.

The matrix A is usually obtained by simulation from a brain modelling by boundary or finite elements. Generally speaking, each term A(i,j) of matrix A represents the measured signal i corresponding to a unit electrical activity in the elementary area j. Thus, the cortex is divided into M elementary areas and each element of the vector x being representative of the electric current density in a voxel, for example taken equal to the norm of the electric current density vector in the voxel in question.

Locating the brain activity amounts to researching the vector x (or, at the very least a vector x insofar as the system (1) is under-determined), from the vector y, that is inverting the relationship (1). For this reason, locating the brain activity from the vector of signals y is sometimes referred to as the “inverse problem” in literature. This inversion is delicate since there is actually an infinity of solutions due to the under-determination of the system (1), the number of sources being considerably higher than the number of sensors. We are then led to make additional hypotheses in order to be able to perform the inversion.

Different solutions to the inverse problem have been suggested in literature, especially the MNE (Minimum Norm Estimate) method, the dSPM (dynamic Statistical Parameter Mapping) method used in locating deep sources, the LORETA (Low Resolution Electromagnetic Tomography) method, beamforming methods especially described in the article by A. Fuchs entitled “Beamforming and its application to brain connectivity” published in the book “Handbook of Brain Connectivity”, V. K. Jirsa, R. A. McIntosh, Springer Verlag, Berlin, pp. 357-378 (2007).

A review of the different abovementioned locating methods can be found in the article by O. Hauk et al. entitled “Comparison of noise-normalized minimum norm estimates of MEG analysis using multiple resolution metrics” published in Neuroimage, Vol. 54, 2011, pp. 1966-1974.

Assuming that the noise is Gaussian and more precisely that the noise samples are independent centred Gaussian random variables which are identically distributed, the solution to (1) can be given by the matrix W which minimizes the square error:

e=∥Wy−x∥² (2)

The equation (2) can also be written as:

e=∥Mx∥²+|Wb∥²=Tr(MRM^{T})+Tr(WCW^{T}) (3)

where M=WA−I_{M}, I_{M} is the identity matrix of a size M×M, Tr is the trace function, R is the covariance matrix of the source signals, and C is the noise covariance matrix. Minimizing the square error e leads to the solution:

W=RA^{T}(ARA^{T}+C)⁻¹ (4)

and therefore to estimating the location of the brain activity given by:

\hat{x}=RA^{T}(ARA^{T}+C)⁻¹y (5)

The noise covariance matrix can be written as C=σ²I_{N} where σ² is the noise variance and I_{N} is the unit matrix of a size N×N. Similarly, if the different sources are considered as being independent and identically distributed (same strength for all the dipoles), there is R=p·I_{M} where p is the strength of the source signal in all the elementary areas, the relationship (5) can be simplified as:

\hat{X}=A^{T}(AA^{T}+λI_{N})⁻¹y (6)

where λ=σ²/p is an adjustment parameter expressing the significance of the noise to the source signal. The expression (6) is the solution of the abovementioned MNE method.

Whatever the locating method considered, the obtained locating accuracy quickly decreases with the signal-to-noise ratio (λ⁻¹). To overcome this difficulty, the article by Liu et al. entitled “Spatiotemporal imaging of human brain activity using functional MRI constrained magnetoencephalography data: Monte Carlo simulations” published in Proceedings of the National Academy of Sciences of the United States of America, vol. 95, n° 15, pp. 8945-8950, July 1998, suggests in particular to use fMRI locating data in order to improve the accuracy of MEG or EEG brain activity localisation. However, this accuracy improvement by data hybridization implies, on the one hand, that an fMRI is available for the same task, which is not worth considering for the direct neural control, and, on the other hand, that the electrical/magnetic activity in the brain is correlated with the hemodynamic response. Consequently, it inevitably introduces a bias by orienting the localisation of the MEG or EEG brain activity towards sources which have been detected by fMRI.

The problem underlying the present invention is consequently to provide a method for estimating the associated brain activity, from psychological signals, in particular magnetoencephalographic or electroencephalographic signals, which has, in some predetermined areas of the cortex, considered as areas of interest, an improved accuracy in relation to other areas of the gridding. This allows to obtain a better estimation of the neural activity, and its time evolution, in areas of the brain which are intended to be targeted by a particular treatment, for example accommodating cortical electrodes.

### DISCLOSURE OF THE INVENTION

The present invention is defined by a method for estimating the electrical activity within a tissue of a subject, wherein:
the tissue is decomposed into a plurality of elementary areas and a transition matrix connecting the electrical activity in each area to a physiological signal around the tissue is determined,
acquiring a plurality of physiological signals is performed thanks to a plurality of sensors disposed around the tissue,
elementary areas of interest are defined among the elementary areas, the number of elementary areas of interest being strictly lower than the number of elementary areas,
a source covariance matrix is established, so that the terms of its diagonal, corresponding to the elementary areas of interest, are lower than the other terms of its diagonal,
the electrical activity in at least one elementary area is estimated (150, 250) from the physiological signals, the source covariance matrix and the noise covariance matrix.

Each elementary area of interest may correspond to a position at which a cortical electrode is intended to be fixed, the method then enabling the electrical activity likely to be measured by said electrode, as well as its time evolution, to be estimated.

The physiological signals can be measured by a non-invasive measuring method, for example by magnetoencephalography or electroencephalography.

The estimation can be based on an MNE criterion.

The noise covariance matrix can be advantageously calculated as the covariance matrix of the physiological signals over a time window during which the subject is at rest.

The electrical activity can then be estimated by the relationship \hat{x}=\tilde{R}A^{T}(A\tilde{R}M^{T}+C)⁻¹y where \hat{x} is a vector representing the electrical activity in the different elementary areas, y is a vector representing the physiological signals acquired by the sensors, A is a matrix estimating the signal measured at each sensor for unit power sources situated in each elementary area, C is the noise covariance matrix and \tilde{R} is the source covariance matrix.

The source covariance matrix \tilde{R} can be established in the following way:
\tilde{R}(i, j)=0 if i≠j, each elementary source being considered as independent from each other,
\tilde{R}(i, i)=λ′ when the elementary area i is an elementary area of interest
\tilde{R}(i, i)=λ when the elementary area i is not an elementary area of interest,
   λ and λ′ being strictly positive real numbers, with λ′<λ.

It is not necessary for all the terms of the diagonal of the source covariance matrix, corresponding to the elementary areas of interest, to have the same value λ′. Similarly, it is not necessary for all the terms of the diagonal, corresponding to the elementary areas which are not elementary areas of interest to have the same value λ. The source covariance matrix \tilde{R} can be established in this case in the following way:
\tilde{R}(i, j)=λᵢⱼ if i≠j, the distinct elementary sources being considered as independent from each other, λᵢⱼ can be equal, for example, to 0 for all the coefficients \tilde{R}(i, j) with i≠j.
\tilde{R}(i, i)=λᵢ′ when the elementary area i is an elementary area of interest, the value λᵢ′ depending on the index i
\tilde{R}(i, i)=λᵢ when the elementary area i is not an elementary area of interest, the value λᵢ depending on the index i.

Each term λᵢ or λᵢ′ being strictly positive with real numbers associated with the elementary area i, each term λᵢ′ associated with an elementary area of interest being lower than the terms λᵢ associated with the other elementary areas. A hierarchy is then established in the elementary area of interest and in the elementary areas outside the elementary area of interest, this hierarchy being governed by λᵢ′ or λᵢ.

According to a second embodiment, the electrical activity is associated with a task performed, imagined, or made by the subject when the latter receives a stimulus, the electrical activity being then measured during a time period following said stimulus, the method then including the following steps:
calculating the correlation coefficients between the different physiological signals and a signal representative of said stimulus
weighting of the coefficients of the covariance matrix, using the correlation coefficients, so as to penalize, in terms of signal-to-noise ratio, the physiological signals which are weakly correlated with the stimulus, the penalizing diminishing the terms of the covariance matrix related to the signals weakly correlated with the stimulus.

In particular, the estimation is based on an MNE-type criterion, and preferably, the covariance matrix of the physiological signals is a noise covariance matrix, calculated over a time window where the stimulus is absent, the electrical activity in each elementary area being then estimated according to the expression \hat{x}(t)=\tilde{R}A^{T}(A\tilde{R}A^{T}+\tilde{C}(t))⁻¹y(t) where \hat{x}(t) is a vector representing the electrical activity at the time t in the different elementary areas, y(t) is a vector representing the physiological signals acquired by the sensors at the time t, A is a matrix estimating the signal measured at each sensor for the unit power sources situated in each elementary area, \tilde{C}(t) is the noise covariance matrix at the time t, and \tilde{R} is the source covariance matrix.

Advantageously, the coefficients of the weighted noise covariance matrix are obtained from the noise covariance matrix by means of the following relationship:

$${\tilde{C}}_{ij} ( t ) = {C}_{ij} for i = 1 , … , N , j = 1 , … , N , i \neq j$$

$${\tilde{C}}_{ii} ( t ) = \frac{{C}_{ii}}{1 + {γ}^{2} {χ}_{i}^{2} ( t )} for i = 1 , … , N$$

where the coefficients Cᵢⱼ(t), i=1, . . . , N, j=1, . . . , N are the coefficients of the weighted noise covariance matrix, the coefficients Cᵢⱼ, i=1, . . . , N, j=1, . . . , N are the coefficients of the noise covariance matrix, N is the number of sensors, y is a predetermined real constant, and χᵢ(t), i=1, . . . N are the correlation coefficients of the physiological signals acquired by the different sensors with the signal representative of the stimulus.

The correlation coefficients χᵢ(t), i=1, . . . , N can be subjected to a normalization prior to weighting the coefficients of the noise covariance matrix.

The correlation coefficients can be obtained by forming a time-frequency or time-scale transform of each physiological signal in order to obtain a plurality of frequency components (Y_{f}(t)) of this signal as a function of time, by calculating the Pearson coefficients (R_{f}(t)) between said frequency components and the signal representative of the stimulus, the correlation coefficient (χ(t)) related to a physiological signal being determined from said obtained Pearson coefficients for this signal.

The correlation coefficient (χ(t)) related to a physiological signal can then be obtained as the extreme value of the Pearson coefficients for the different frequency components of this signal.

One object of the invention is also a method for defining the position of at least one cortical electrode, the method including:
estimating the electrical activity, such as above described, by considering, especially successively, different sets of elementary areas of interest, each set of elementary area of interest including at least one elementary area of interest, so as to obtain an estimation of the electrical activity corresponding to each considered set of elementary areas,
comparing the estimation of the electrical activity for each of the different sets of elementary areas of interest,
defining the optimum position of each cortical electrode as a function of this comparison.

The aim of the comparison is essentially to determine the optimum position of each electrode from the point of view of the signal-to-noise ratio.

### BRIEF DESCRIPTION OF THE DRAWINGS

Further characteristics and advantages of the invention will appear upon reading a preferential embodiment of the invention made with reference to the appended figures among which:

FIG. 1 schematically represents the flow chart of a method for estimating the electrical activity in a tissue according to a first embodiment of the invention;

FIG. 2 schematically represents the flow chart of a method for estimating the electrical activity in a tissue according to a second embodiment of the invention;

FIG. 3 schematically represents an exemplary calculation of a correlation coefficient between a physiological signal and a stimulus signal.

### DETAILED DISCLOSURE OF PARTICULAR EMBODIMENTS

A system for acquiring physiological signals from a plurality of sensors disposed around a tissue of interest of a human or animal subject will be thereafter considered, this tissue being the seat of an electrical activity when this subject performs, sees, or visually imagines a task. For the sake of illustration and without a generalization prejudice, we will more particularly consider the case of a magnetoencephalographic acquisition system, being understood that other acquisition systems can be alternatively used, especially an electroencephalographic acquisition system.

The magnetoencephalographic system comprises, in a known manner, a “MEG helmet” placed at a few centimetres from the subject's cranium. This helmet comprises a plurality of sensors situated in different points; each sensor can be made of one or more simple sensors. The simple sensors can be precision magnetometers and planar (or axial) gradiometers. Alternatively, they can be radial gradiometers such as those described in the article by J. Vrba et al. entitled “Signal processing in magnetoencephalography”, Methods 25, 249-271 (2001). The motions of the subject's head are furthermore recorded and compensated thanks to coils placed in stationary points with respect to the subject's head and generating a magnetic field in a frequency band far from that of the MEG signal.

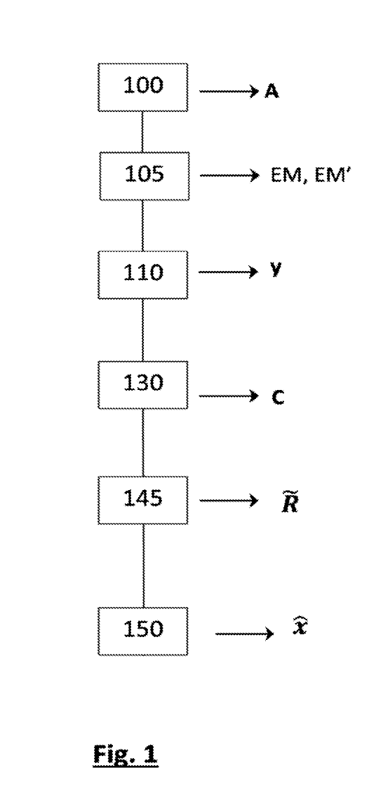

FIG. 1 schematically represents a method for estimating the electrical activity within a subject's tissue, according to a first embodiment of the invention.

In step 100, thanks to the gridding, M elementary areas are defined on the observed tissue, each elementary area being represented by a point Pₘ, with 1≦m≦M. As previously mentioned, this point can be the centre or a vertex of a square. The lead field matrix A, conventionally referred to as a gain matrix (dimension N×M) coming from the resolution of the direct problem is determined, each element Aᵢⱼ of which represents the signal measured by the sensor i corresponding to the unit electrical activity in the elementary area j (or at a point Pj associated with the elementary area j). The lead field matrix A is obtained by simulation, for example prior to acquiring physiological signals, by estimating signals measured by the different sensors for a given value of an elementary source. The process is repeated for the M elementary areas such that the M lines of the matrix A are successively obtained.

In step 105, among the M elementary areas, M′ elementary areas of interest are selected, M′ being a positive integer strictly lower than M. Each elementary area of interest corresponds to an area in which estimating the neuronal activity with an increased accuracy is wanted, with respect to the other elementary areas M.

Each elementary area of interest can correspond to the provided place of a cortical electrode. The cortical surface is for example obtained by an MRI modality, in which case the gridding can be made in a mark associated with the MRI data. The coordinate of each electrode is then determined in this mark, and for each cortical electrode, the nearest elementary area of interest is determined. It amounts to determining, for each electrode, the nearest gridding point Pₘ, this point being then considered as a point of interest.

An elementary area of interest can also correspond to a pathology or lesion area, or to a functional area identified by a functional MRI or PET scan-type modality.

The number M′ of elementary areas of interest is typically of a few tens, and for example between 5 and 100, whereas the number M of considered elementary areas is greater than 1,000, or even greater than 10,000. Finally, this step amounts to forming a sub-assembly EM′ of M′ elementary areas in the set EM grouping the M elementary areas of the gridding.

In step 110, the physiological signals are acquired using sensors disposed at the periphery of the cortex. As previously mentioned, these sensors can be electrodes, magnetometers, or gradiometers. Preferably, these sensors are non-invasive and are disposed outside the body. They can especially be EEG electrodes or MEG-type magnetic sensors. The recording can take place when the subject is at rest, or when the subject performs, imagines, or visualizes a task. The recording can also take place when the subject suffers from a particular neuronal pathology, for example in relation to inter-critical paroxysmal events in the epileptic patient, or in a patient suffering from a neuronal degenerative disease or from a traumatic or tumoral lesion. During this step 110, a vector y, of a size N representative of the signals acquired by the different sensors is formed.

In step 130, the noise covariance matrix C is calculated, each element Cᵢⱼ of this matrix being obtained according to the expression

C(i,j)=E((yᵢ−E(yᵢ))(yⱼ−E(yⱼ))) (7)

where
E(•) means the mathematical expectation,
yᵢ corresponds to the i^{th} component of the vector y representing the measured signal,
   C corresponds to a covariance matrix of physiological signals when the latter correspond to a brain background activity, referred to as a “baseline” activity, considered as noise. The examined subject is then at rest.

In step 145, a weighted source covariance matrix R is established, by assigning, to each term \tilde{R}ᵢᵢ of the diagonal the index of which corresponds to an elementary area of interest (i∈EM′), a value lower than that of a term \tilde{R}ᵢᵢ of the diagonal the index of which does not correspond to an elementary area of interest (i∉M′). Whereas the source signals are re-designed in M points Pₘ, the re-design accuracy is then improved at the elementary areas of interest, or more precisely at each point associated with an elementary area of interest. This can thus be referred to as spatial penalizing, since a penalizing term (λ or λ′), depending on its position on the gridding, is assigned to each gridding point. The source covariance matrix is initialized according to the following expression:
\tilde{R}ᵢᵢ=0 if i≠j, the elementary sources being considered as independent of one another,
\tilde{R}ₒ=λ′ if i∉EM′

\tilde{R}ᵢᵢ=λ if i∈EM′ (8)

with λ′<λ, and preferably λ′≦10λ or even λ′≦100λ and λ′ being two strictly positive real numbers.

This amounts to a priori diminishing the part of noise affecting the estimation of the source signal at the elementary areas of interest with respect to the other elementary areas. Thus, generally speaking, the source covariance matrix is initialized so that the terms of its diagonal, corresponding to elementary areas of interest are lower than the terms of its diagonal not corresponding to elementary areas of interest.

In step 140, the electrical activity is estimated in each elementary area of the tissue, by resolving the equation:

\hat{x}=\tilde{R}A^{T}(A\tilde{R}A^{T}+C)⁻¹y (9)

with:
\hat{x}: vector of a dimension M, each term \hat{x}(i) of this vector corresponding to the estimation of the electrical activity at the point Pᵢ representing an elementary area i among the set of the M elementary areas;
\tilde{R}: source covariance matrix (dimension M×M), such as previously defined;
A: lead field matrix;
C: noise covariance matrix, of a dimension (N×N);
y: vector of the physiological signal measurements, of a dimension N.

Those skilled in the art will understand that determining matrices A, C, and \tilde{R} does not necessarily follow the order provided in this first example. For example, the matrix \tilde{R} can be defined prior to the matrix C and/or the matrix A.

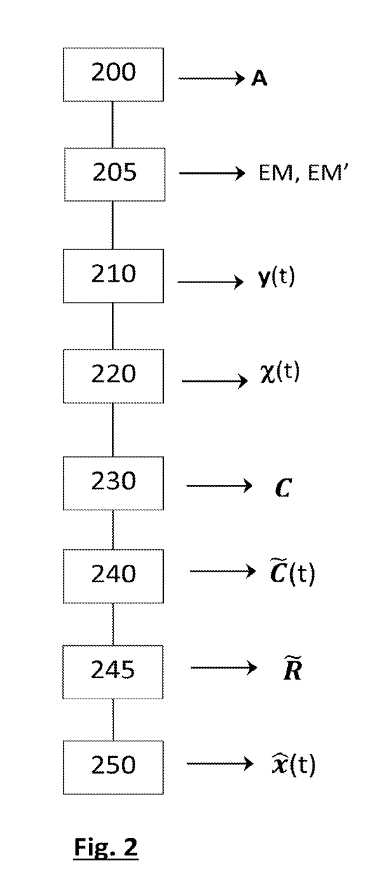

FIG. 2 schematically represents a method for estimating the electrical activity within a subject's tissue, according to a second embodiment of the invention. According to this embodiment, recording the signals takes place when the subject performs, imagines, or visualizes a task, the latter can be represented by a binary variable η(t) indicating a sensory stimulus, for example a visual or auditory stimulus. For example, when the variable η(t) assumes the value 1, the stimulus is applied and when it assumes the value 0, it is not. When the stimulus is applied, the subject performs, imagines or visualizes the task in question.

Steps 200 and 205 are respectively similar to previously described steps 100 and 105.

During step 210, the stimulus can be repeated so as to acquire a plurality of sequences y(t) where y is, as previously defined, the vector (of a dimension N) of the physiological signals acquired by the different sensors at the time t).

In step 220, for each component yₙ(t), n=1, . . . , N, of the vector y(t), a coefficient χₙ representing the correlation between the signal yₙ(t) and the stimulus η(t) is calculated over a given time range, the coefficient χₙ being all the higher in terms of absolute value that the correlation between this component and the stimulus is significant in this time range. The correlation coefficient χₙ can be obtained according to different alternatives, as described later. Generally speaking, the coefficient χₙ depends on the time range considered for calculating the correlation and consequently on the time. For this reason, it will be later noted as χₙ(t).

At the end of the learning phase, a plurality of coefficients χₙ(t), n=1, . . . , N is available, indicating, as a function of time, to which extent the different physiological signals are correlated “with the stimulus”, in other words, are relevant regarding the task in question.

In step 230, the noise covariance matrix C is calculated. This noise covariance matrix is advantageously obtained when no stimulus is applied and when no task is performed by the subject, in this case when η(t)=0. In other words, the component of the covariance matrix is obtained by:

Cᵢⱼ=E[(yᵢ−E(yᵢ))(yⱼ−E(yⱼ))] when η(t)=0 (10)

The mathematical expectation E(Z) can be estimated from the average of Z over the time interval during which the stimulus is absent.

This embodiment includes a step 240, during which the coefficients (here the diagonal terms) of the noise covariance matrix are weighted by means of the abovementioned correlation coefficients, so as to penalize, in terms of signal-to-noise ratio, the physiological signals having a weak correlation with this stimulus. This weighting correlatively promotes, in terms of signal-to-noise ratio, the physiological signals having a high correlation with the stimulus. Penalizing results in increased coefficients of the covariance matrix related to the physiological signals weakly correlated with the stimulus, insofar as the covariance matrix is that of the noise covariance matrix.

This weighting is dynamic insofar as the weighting coefficients vary as a function of time. The result of this weighting is a weighted covariance matrix noted as Ĉ(t), which evolves over time. For example, the coefficients of the matrix Ĉ(t) can be obtained from the coefficients of the noise covariance matrix C, in the following way:

$${\tilde{C}}_{ij} ( t ) = {C}_{ij} for i = 1 , … , N , j = 1 , … , N , i \neq j \\ {\tilde{C}}_{ii} ( t ) = \frac{{C}_{ii}}{1 + {γ}^{2} {χ}_{i}^{2} ( t )} for i = 1 , … , N \qquad ( 11 )$$

where γ is a predetermined coefficient. Advantageously, the correlation coefficients χᵢ(t), i=1, . . . , N are normalized:

$${χ}_{i} ( t ) = \frac{{χ}_{i} ( t ) - min ( {χ}_{i} )}{max ( {χ}_{i} ) - min ( {χ}_{i} )} \qquad ( 12 )$$

so that they take their values in the interval [0,1]. Other weighting functions can be considered by those skilled in the art without departing from the scope of the present invention.

Step 245 is similar to step 145 of the previous embodiment.

In step 150, an estimation of the electrical activity in the tissue is performed at each moment from the vector of the physiological signals y, of the lead field matrix A, as well as the weighting noise covariance matrix, \tilde{C}:

\hat{x}(t)=\hat{R}A^{T}(A\tilde{R}A^{T}+Ĉ(t))⁻¹y(t) (13)

The expression (13) implies the inversion of a matrix at each considered time range. In practice, merely performing this inversion every N_{f} time window (N_{f} being an integer greater than 1) can be enough, by replacing in the expression (8) the correlation coefficients by their respective averages over N_{f} time windows.

It should be noted that according to the first embodiment, estimating the source activity is performed by implementing a spatially weighted source covariance matrix, insofar as weighting is performed as a function of the localisation of each source. In the second embodiment, the spatially weighted source covariance matrix is combined with a noise covariance matrix in the sensor space, the noise covariance matrix being weighted as a function of the time correlation between the measured signals and the performed task.

In any case, the thus-estimated electrical activity can be represented as an image to locate the activity or can be processed, in the case of a brain activity, in order to generate a direct neural control. In the latter case, the processing in question can comprise the integration of the module of the vector \hat{x} on a predetermined area of the brain and the comparison of the integration result with a threshold, or also a spatial correlation of the vector \hat{x} with a predetermined pattern.

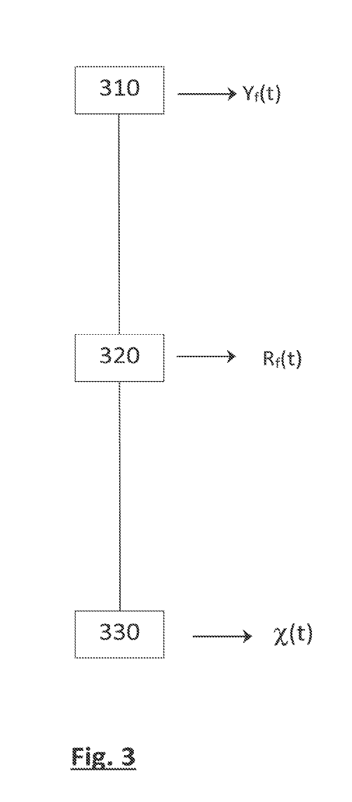

FIG. 3 schematically represents an exemplary calculation of the correlation coefficient of a physiological signal with a stimulus signal, that is, more precisely, of a signal yₙ(t) provided by a sensor and the stimulus η(t), such as above-defined. The physiological signal yₙ(t) will be simply denoted y(t) in the following, the calculation being identical whatever the sensor.

In a first step, 310, a time-frequency transform or a time-scale transform of the physiological signal y(t) is calculated. The time-frequency transform can be for example a weighting short-term Fourier transform using a sliding time window, the time-scale transform can be a continuous wavelet transform (CWT) in a manner know per se. The Morlet-Gabor wavelet or a so-called Mexican hat wavelet can be used to this end.

In any case, a frequency representation as a function of time, Y_{f}(t), of the physiological signal y(t), is obtained the term Y_{f}(t) giving the “instant” frequency component (or more precisely in a frequency band) of the signal y(t).

If need be, these frequency components can be smoothed over time by a low-pass filtering, for example by means of a moving average with a forgetting coefficient.

In a second step 320, the Pearson coefficient R_{f} of each frequency component Y_{f} is calculated in the following way:

$${R}_{f} ( t ) = \frac{1}{{σ}_{η} \cdot {σ}_{{Y}_{f}}} {\int }_{[ t , t + T ]} {Y}_{f} ( u ) ( η ( u ) - \overline{η} ) d u \qquad ( 14 )$$

the integration over the sliding window [t,t+T] can of course be performed by means of a discrete summation and the calculation being performed for a discrete set of the frequency. σ_{η} and σ_{Y}_{_{f}} respectively represent the variance stimulus of η and of the frequency component Y_{f} and η̅ is the average value of η on the sliding window in question.

In a third step 330, the correlation coefficient χ(t) of the physiological signal y(t) with the stimulus η(t) is calculated from the Pearson coefficients R_{f}(t). For example, for χ(t), the extreme value of R_{f}(t) in the frequency range of interest can be taken:

$$χ ( t ) = {extrem}_{f} [ {R}_{f} ( t ) ] \qquad ( 15 )$$

It is noted that the extreme value of a function is that of the maximum value and of the minimum value which is the largest in absolute value. Similarly, since it intervenes previously only in absolute value, the correlation coefficient can be chosen as the maximum of |R_{f}(t)| in the frequency range of interest. Other calculation alternatives of the correlation coefficient can be considered by those skilled in the art, for example the integration of |R_{f}=(t)| or of (R_{f}(t))² on the frequency range of interest.

Whatever the embodiment, establishing the source covariance matrix \tilde{R} such as previously described enables the neuronal signals at particular points to be simulated with an increased accuracy, and this prior to introducing invasive means, in particular cortical electrodes. Such simulations enable the placing of electrodes to be optimized, by identifying, by a non-invasive method (MEG, EEG) the optimum points of the cortical surface from the point of view of the electrical cortical activity, compared with a targeted application. It can be for example points, at which the rebuilt cortical signal has a good correlation with a given task. It can also be points at which the reactivity of the alpha rhythm is particularly high.

1. A method for estimating the electrical activity within a tissue of a subject, wherein
the tissue is decomposed into a plurality of elementary areas and a transition matrix connecting the electrical activity in each area to a physiological signal around the tissue is determined,
acquiring a plurality of physiological signals is performed thanks to a plurality of sensors disposed around the tissue,
a noise covariance matrix is estimated;
said method being characterised in that:
elementary areas interest are defined among the elementary areas, the number of elementary areas of interest being strictly lower than the number of elementary areas,
a source covariance matrix is established, so that the terms of its diagonal, corresponding to the elementary areas of interest, are lower than the other terms of its diagonal,
the electrical activity in at least one elementary area is estimated from the physiological signals, the source covariance matrix and the noise covariance matrix

2. The method for estimating the electrical activity within a tissue according to claim 1, characterised in that the estimation is based on an MNE criterion.

3. The method for estimating the electrical activity within a tissue according to claim 2, characterised in that the noise matrix is calculated as the covariance matrix of the physiological signals over a time window in which the subject is at rest.

4. The method for estimating the electrical activity within a tissue according to claim 2, characterised in that the electrical activity in each elementary area is estimated by means of:
\hat{x}=\tilde{R}A^{T}(A\tilde{R}A^{T}+C)⁻¹y
where \hat{x} is a vector representing the electrical activity in the different elementary areas, y is a vector representing the physiological signals acquired by the sensors, A is a matrix estimating the signal measured at each sensor for unitary power sources situated in each elementary area, C is the noise covariance matrix, and \tilde{R} is the source covariance matrix.

5. The method for estimating the electrical activity within a tissue according to claim 1, according to which the source covariance matrix is established by means of:
\tilde{R}ᵢⱼ=0 if i≠j, the elementary sources being considered as independent of one another,
\tilde{R}ᵢᵢ=λ′, when the elementary area i is an elementary area of interest,
\tilde{R}ᵢᵢ=λ, when the elementary area i is not an elementary area of interest,
λ et λ′ being strictly positive real numbers, with λ′<λ,

6. The method for estimating the electrical activity within a tissue according to claim 1, according to which the electrical activity is associated with a task performed, imagined, or made by the subject when the latter receives a stimulus, the electrical activity being then measured during a time period following said stimulus, the method including the following steps:
calculating the correlation coefficients between different physiological signals and a signal representative of said stimulus
weighting the coefficients of the covariance matrix of the physiological signals, using the correlation coefficients, so as to penalize, in terms of signal-to-noise ratio, the physiological signals which are weakly correlated with the stimulus, the penalizing diminishing the terms of the correlation matrix related to the signals weakly correlated with the stimulus.

7. The method for estimating the electrical activity within a tissue according to claim 6, according to which the estimation is based on an MNE-type criterion, characterised in that the covariance matrix of the physiological signals is a noise covariance matrix calculated over a time window where the stimulus is absent and that the electrical activity in each elementary area is estimated by means of:
\hat{x}(t)=\tilde{R}A^{T}(A\tilde{R}A^{T}+\tilde{C}(t))⁻¹y(t)
where \hat{x}(t) is a vector representing the electrical activity at the time t in the different elementary areas, y(t) is a vector representing the physiological signals acquired by the sensors at the time t, A is a matrix estimating the signal measured at each sensor for unit power sources situated in each elementary area, \tilde{C}(t) is the noise covariance matrix at the time t and \tilde{R} is the source covariance matrix.

8. The method for estimating the electrical activity within a tissue according to claim 7, characterised in that the coefficients of the weighted noise covariance matrix are obtained from the noise covariance matrix by means of the following relationship:
$${\tilde{C}}_{ij} ( t ) = {C}_{ij} for i = 1 , … , N , j = 1 , … , N , i \neq j$$
$${\tilde{C}}_{ii} ( t ) = \frac{{C}_{ii}}{1 + {γ}^{2} {χ}_{i}^{2} ( t )} for i = 1 , … , N$$
where the coefficients \tilde{C}ᵢⱼ(t), i=1, . . . , N, j=1, . . . , N are the coefficients of the weighted noise covariance matrix, the coefficients Cᵢⱼ, i=1, . . . , N, j=1, . . . , N are the coefficients of the noise covariance matrix, N is the number of sensors, γ is a predetermined real constant and χᵢ(t), i=1, . . . , N are the correlation coefficients of the physiological signals acquired by the different sensors with the signals representative of the stimulus.

9. The method for estimating the electrical activity within a tissue according to claim 8, characterised in that the correlation coefficients χᵢ(t), i=1, . . . , N are subjected to a normalization prior to the coefficient weighting of the noise covariance matrix.

10. The method for estimating the electrical activity within a tissue according to claim 9, characterised in that the correlation coefficients are obtained by performing a time-frequency or time-scale transform of each physiological signal in order to obtain a plurality of frequency components (Y_{f}(t) of this signal as a function of time, by calculating the Pearson coefficients (R_{f}(t)) between said frequency components and the signal representative of the stimulus, the correlation coefficient
(χ(t)) related to a physiological signal being determined from said Pearson coefficients obtained for this signal.

11. The method for estimating the electrical activity within a tissue according to claim 10, characterised in that the correlation coefficient (χ(t)) related to a physiological signal is obtained as the extreme value of the Pearson coefficients for the different frequency components of this signal.
